(19) United States
(12) Patent Application Publication
(10) Pub. No.: US 2005/0221281 A1
(43) Pub. Date: Oct. 6, 2005

(54) Self-contained microfluidic biochip and apparatus

(76) Inventor: Winston Z. Ho, Hacienda Heights, CA (US)

Correspondence Address: Winston Z. Ho, 14541 Langhill Drive, Hacienda Heights, CA, 91745, US

(21) Appl. No.: 11/117,908

(22) Filed: Apr. 29, 2005

Related U.S. Application Data
(63) Continuation-in-part of application No. 11/013,609, filed on Dec. 16, 2004.
Application No. 11/013,609 is a continuation-in-part of application No. 10/338,451, filed on Jan. 8, 2003.

(51) Int. Cl. C12Q 1/00; C12M 1/34

(52) U.S. Cl. 435/4; 435/287.1

(57) ABSTRACT

A biochip and apparatus is disclosed for performing biological assays in a self-contained microfluidic platform. The disposable biochip for multi-step reactions comprises a body structure with a plurality of reagent cavities and reaction wells connected via microfluidic channels; the reagent cavities with reagent sealing means for storing a plurality of reagents; the reagent sealing means being breakable and allowing a sequence of reagents to be released into microfluidic channel and reaction well; and the reaction well allowing multi-step reactions to occur. The apparatus may further comprise a microactuator, a heating and cooling element, a detector, a moving stage, a magnetic field generator, and a processor operable to perform all necessary functions, such as reagent delivery, magnetic purification, mixing and incubation, heating and cooling, and optical detection on a microfluidic biochip.

### CROSS-REFERENCE TO RELATED APPLICATIONS

This application is a continuation-in-part application of U.S. patent application Ser. No. 11/013,609, filed Dec. 16, 2004, which is a continuation-in-part application of U.S. patent application Ser. No. 10/338,451, filed Jan. 8, 2003. Both applications are incorporated herein by reference in their entireties for all purposes.

### FIELD OF THE INVENTION

The invention is related to a method using a self-contained biochip that is preloaded with necessary reagents, and utilizes microfluidic and pressure-driven microactuator mechanisms to perform biological reactions and assays, molecular diagnostics, sample preparation, nucleic acid extraction, gene expression profiling or screening of candidate genes for a genetic study. The biochip analytical apparatus is configured rapidly and automatically performing multiple-step bioprocess and measuring the quantities of chemical and biological species in a sample.

### BACKGROUND OF THE INVENTION

Current hospital and clinical laboratories are facilitated with highly sophisticated and automated systems with the capability to run up to several thousand samples per day. These high throughput systems have automatic robotic arms, pumps, tubes, reservoirs, and conveying belts to sequentially move tubes to proper position, deliver the reagents from storage reservoirs to test tubes, perform mixing, pump out the solutions to waste bottles, and transport the tubes on a conveyer to various modules. Typically three to five bottles of about 1 gallon per bottle of reagent solutions are required. While the systems are well proved and accepted in a laboratory, they are either located far from the patients or only operated once numerous samples have been collected. Thus, it often takes hours or even days for a patient to know their test results. These systems are very expensive to acquire and operate and too large to be used in point-of-care testing setting.

The biochips offer the possibility to rapidly and easily perform multiple biological and chemical tests using very small volume of reagents in a very small platform. In the biochip platform, there are a couple of ways to deliver reagent solutions to reaction sites. The first approach is to use external pumps and tubes to transfer reagents from external reservoirs. The method provides high throughput capability, but connecting external macroscopic tubes to microscopic microchannel of a biochip is challenging and troublesome. The other approach is to use on-chip or off-chip electromechanical mechanisms to transfer self-contained or preloaded reagents on the chips to sensing sites. While on-chip electromechanical device is very attractive, fabricating micro components on a chip is still very costly, especially for disposable chips. On the other hand, the off-chip electromechanical components, facilitated in an analytical apparatus, that are able to operate continuously for a long period of time is most suited for disposable biochip applications.

U.S. Patent Application publication 2002/0124879, entire contents of which are incorporated herein by reference, discloses a device for moving a fluid in a fluidics system. The device includes one or more controllably openable closed chambers. The pressure within the closed chambers is lower than the ambient pressure outside the fluidics system or lower than the pressure within another channel of the fluidics system. The closed chamber is configured for being controllably opened. The chamber is configured such that when a chamber is opened the chamber is in fluidic communication with a flow channel included within the fluidics system. The fluid may be moved into the flow channel or may be moved within the flow channel. The fluid may be a liquid, a gas, a mixture of gases or an aerosol. The fluidics system may include a controller for controlling the opening of a selected chamber or chambers.

U.S. Patent Application publication 2002/0187560, entire contents of which are incorporated herein by reference, discloses a microfluidic device capable of combining discrete fluid volumes generally including channels for supplying different fluids toward a sample chamber and means for establishing fluid communication between the fluids within the chamber. Certain embodiments utilize adjacent chambers or subchambers divided by a rupture region such as a frangible seal. Further embodiments utilize one or more deformable membranes and/or porous regions to direct fluid flow. Certain devices may be pneumatically or magnetically actuated.

European Patent No. 1203959, entire contents of which are incorporated herein by reference, discloses an analyzing cartridge for use in analysis of a trace amount of sample, enabling analysis and detection to be carried out conveniently, and a method for producing the same. The invention also provides an analysis method using the analyzing cartridge and a liquid feed control device that is attached to the analyzing cartridge, and controls the feeding of liquid in the analyzing cartridge.

The microfluidics-based biochips have broad application in fields of biotechnology, molecular biology, and clinical diagnostics. The self-contained biochip, configured and adapted for insertion into an analytical apparatus, provides the advantages of compact integration, ready for use, simple operation, and rapid testing. For microfluidic biochip manufacturers, however, there are two daunting challenge. One of the challenges is to store reagents without losing their volumes over product shelf life. The storage cavity should have a highly reliable sealing means to ensure no leak of reagent liquid and vapor.

Although many microscale gates and valves are commercially available to control the flow and prohibit liquid leakage before use, they are usually not hermetic seal for the vaporized gas molecules. Vapor can diffuse from cavity into microchannel network, and lead to reagent loss and cross contamination. The second challenge is to deliver a very small amount of reagents to a reaction site for quantitative assay. The common problems associated are air bubbles and dead volume in the microchannel system. An air bubble forms when a small channel is merged with a large channel or large reaction area, or vice versa. Pressure drops cause bubble formation. The air bubble or dead volume in the microfluidic channel can easily result in unacceptable error for biological assay or clinical diagnosis.

Several prior art devices have been described for the performance of a number of microfluidics-based biochip and analytical systems. U.S. Pat. No. 5,096,669 discloses a disposable sensing device with special sample collection means for real time fluid analysis. The cartridge is designed for one-step electrical conductivity measurement with a pair of electrodes, and is not designed for multi-step reaction applications. U.S. Pat. No. 6,238,538 to Caliper Technologies Corp. discloses a method of using electro-osmotic force to control fluid movement. The microfabricated substrates are not used for reagent storage. U.S. Pat. No. 6,429,025 discloses a biochip body structure comprising at least two intersecting microchannels, which source is coupled to the least one of the two microchannels via a capillary or microchannel. Although many prior art patents are related to microfluidic platform, none of them discloses liquid sealed mechanism for self-contained biochips. They are generally not designed for multi-step reactions application.

Furthermore, since the advent of microarrays in the mid 1980s, investigators have continued to manually perform individual steps in microarray experiments, including DNA/RNA probe synthesis, labeling, hybridization, post-hybridization washes, drying, and scanning. Microarrays are generally printed on 25 mm×75 mm microscopic glass or plastic slides. Hybridization is performed using a glass cover slide or dedicated chambers, whereas subsequent washing and drying of the slides takes place in separate dishes. Unfortunately current microarray systems suffer from the consumption of massive amounts of material and time, and from run-to-run variations in data as a result of error prone manual steps (about 15-20 steps). Manual steps may include pipetting of reagents into different tubes and transfer of reagents between tubes and steps, which also increases the risk of contamination. Moreover, inconsistent data might not be corrected by sophisticated normalization algorithms, thus leading to misinterpretations of the underlying biology.

DNA microarrays, especially for expression profiling, have proven to be a very useful tool in molecular biology. There is no doubt about the impact of this method on implementing more accurate and faster diagnostics of complex genetic diseases, like cancer and immunological disorders, and fostering the development of more specific drugs and therapies that are tailored to patient subtypes. Clinical diagnostics is extremely dependent on the confidence of data replication. The disadvantages of the prior microarray systems may include the following, such as low confidence as a research or diagnostic tool due to the high risk of contamination, inconsistency of data due to a large number of error prone manual handling steps and solution transfers, inconvenient and time consuming manual processing steps, large modular or bench top instruments that are not easily accessible or portable, complicated operational procedures that demand highly trained personnel, or requiring the use of a relatively expensive platform.

U.S. Pat. No. 6,618,679, entire contents of which are incorporated herein by reference, discloses methods, compositions and kits for gene expression analysis and gene expression profiling. The method for analyzing gene expression comprises: a) obtaining a plurality of target sequences, wherein the plurality of target sequences comprises cDNA; b) multiplex amplifying the plurality of target sequences, wherein multiplex amplifying comprises combining the plurality of target sequences, a plurality of target-specific primers, and one or more universal primers, and wherein the universal primer is provided in an excess concentration relative to the target-specific primer, thereby producing a plurality of amplification products; c) separating one or more members of the plurality of amplification products; d) detecting one or more members of the plurality of amplification products, thereby generating a set of gene expression data; e) storing the set of gene expression data in a database; and f) performing a comparative analysis on the set of gene expression data, thereby analyzing the gene expression.

U.S. Pat. No. 6,816,790, entire contents of which are incorporated herein by reference, discloses a method for determining a concentration level of a target nucleic acid, the target nucleic acid comprising at least one target oligonucleotide. The method determines (i) a measure of affinity value of the target oligonucleotide with a probe oligonucleotide; and (ii) a hybridization intensity value for the target oligonucleotide and the probe oligonucleotide at a probe spot. The measure of affinity value and the hybridization intensity value are used to determine the concentration level of the target nucleic acid.

Current molecular diagnostic systems are very large, bulky, and weights more than 1,200 lb., such as Gen-Probe's Tigris DTS system (San Diego, Calif.). The operation procedure involving multi-step reaction is very complicated. The microfluidic biochip system automates all the steps of sample processing from target (pathogen rRNA) capture over target amplification to target detection in a space-saving 12″×12″×10″ platform and can be operated in a programmed walk-away fashion.

### SUMMARY OF THE INVENTION

In accordance with preferred embodiments of the present invention, a self-contained microfluidic disposable biochip is provided for performing a variety of chemical and biological analyses. The disposable biochip is constructed with the ability of easy implementation and convenient storage of necessary reagents over the reagent product shelf life without loss of volume.

Another object of this invention is to provide a ready to use, highly sensitive and reliable biochip. Loading a sample and inserting it into a reading apparatus are the only required procedures. All the commercially available point of care testing (POCT) analyzers have poor sensitivity and reliability in comparison with the large laboratory systems. The key problem associated with a POCT is the variation in each step of reagent delivery during multiple-step reactions. Especially, the problems occur in closed confinement. For example, a common sandwiched immunoassay, three to six reaction steps are required depending on the assay protocol and washing process. Each reaction requires accurate and reproducible fluids volume delivery.

Another object of this invention is to provide the ability of a biochip with the flexibility for performing a variety of multi-step chemical and biological measurements. The disposable biochip is configured and constructed to have the number of reagent cavities matching the number of assay reagents, and the analytical apparatus performs multiple reactions, one by one, according to the assay protocol.

Another object of this invention is to provide a biochip that can perform multi-analyte and multi-sample tests simultaneously. A network of microfluidic channel offers the ability to process multiple samples or multiple analytes in parallel.

Another object of this invention is to mitigate the problems associated with air bubble and dead volume in the microchannel. The air bubble or dead volume in the microfluidic channel easily results in unacceptable error for biological assay or clinical diagnosis. This invention is based on a microfluidic system with a reaction well, which has an open volume structure and eliminates the common microfluidic problems.

The present invention with preloaded biochips has the advantages of simple and easy operation. The resulting analytical apparatus provides accurate and reproducible results. It should be understood, however, that the detailed description and specific examples, while indicating preferred embodiments of the present invention, are given by way of illustration and not of limitation. Further, as is will become apparent to those skilled in the area, the teaching of the present invention can be applied to devices for measuring the concentration of a variety of liquid samples.

Some aspects of the invention relate to a method for analyzing gene expression profiling comprising steps of: a) procurement means for simultaneous nucleic acid synthesis; b) treatment means for nucleic acid amplification and labeling of at least one nucleic acid element; and c) analyzing means for at least single-color hybridization to an integrated array of spotted bio-molecules (such as nucleic acids, peptides, protein-nucleic acids, carbohydrates and lipids).

In a further embodiment, all steps are performed in an apparatus comprising a self-contained disposable microfluidic biochip for performing multi-step reactions, the biochip comprising: a body structure comprising a plurality of reagent cavities and at least one reaction well connected via microfluidic channels, the reagent cavities each storing a reagent and each comprising a breakable seal allowing the reagent to be selectively released into the reaction well upon being punctured. In one embodiment, all steps are performed in the apparatus for out-lab use in a remote area.

In a further embodiment, the microfluidic channels of the biochip have a dimension between about 0.1 μm and 2.0 mm in cross section, preferably a dimension between about 1.0 μm and 50 μm in cross section.

In a further embodiment, the surface of the microfluidic channels of the biochip is treated with a surface tension reducing agent.

In a further embodiment, the reaction well is facilitated with at least one biological probe that is selected from a group consisting of proteins, nucleic acids, receptors, and cells. In one embodiment, the biochip comprises a plurality of reaction wells, each in flow communication with the plurality of reagent cavities.

In a further embodiment, the apparatus further comprises a vacuum suction for removing waste from the reaction well or a sampling port.

In a further embodiment, at least one of the reagent cavities comprises a second reagent stored in a chamber with a second breakable seal, whereby the second reagent flows and interacts with any other reagent in a reagent cavity when the second breakable seal is punctured. In one embodiment, the seal comprises a thin film located at the bottom of each reagent cavity for preventing reagent escape, and wherein each reagent cavity comprises a microcap assembly located at the top of each reagent cavity, a pin being provided at adjacent the film configured for puncturing the film. In another embodiment, the apparatus further comprises a microactuator, wherein the microactuator and the biochip are supported for movement relative to each other adapted for positioning the microactuator at each of the microcap assembly, wherein the microactuator is structured and configured to deliver a downward pressure to the microcap assembly.

Some aspects of the invention relate to a method of screening of candidate genes for a genetic study comprising steps of: a) procurement means for simultaneous nucleic acid synthesis; b) treatment means for nucleic acid amplification and labeling of at least one nucleic acid element; and c) analyzing means for at least single-color hybridization to an integrated array of spotted bio-molecules (such as nucleic acids, peptides, protein-nucleic acids, carbohydrates and lipids), wherein all three means are performed in a self-contained disposable microfluidic biochip apparatus, the apparatus comprising: a body structure comprising a plurality of reagent cavities and at least one reaction well connected via microfluidic channels, the reagent cavities each storing a reagent and each comprising a breakable seal allowing the reagent to be selectively released into the reaction well upon being punctured.

In a further embodiment, the genetic study is selected from a group consisting of a research of immune and infectious diseases, a research of drug target identification and validation, and a research of identification of a threat agent selected from a group consisting of bacteria, viruses, germs, enzymes, fungi, and combination thereof.

Some aspects of the invention relate to a method for analyzing diseases or biological pathogens in a small biochip platform by employing the existing and well-established chemiluminescence bioassay menu used in large clinical laboratory systems.

In a further embodiment, an analytical apparatus for use with a microfluidic biochip including self-contained reagents and patterned reagents wells, microchannels, and reaction wells, the apparatus comprising a micro mechanical actuator, heating and cooling means for heating or cooling the reagents, a vacuum suction, a magnetic field generator, and a moving stage to perform all necessary functions, wherein the functions include reagent delivery, magnetic purification, mixing and incubation, heating and cooling, and optical detection on the microfluidic biochip.

Some aspects of the invention relate to an analytical apparatus for use with a microfluidic biochip that comprises a plurality of patterned reagent wells with self-contained reagents that are connected to reaction wells via microchannels, the apparatus comprising: (a) a micro mechanical actuator for delivering downward pressure to transport at least one of the reagents from the patterned reagent wells into a first reaction well; (b) a detector for measuring optical properties of fluid in the first reaction well; (c) a moving stage mounted at the biochip, wherein the moving stage is sized and configured to accurately position each of the reagent wells under the actuator, and position the first reaction well above or below the detector; and (d) a processor configured to control the moving stage and process the optical properties.

Some aspects of the invention relate to a method for measuring optical properties of a fluid combining at least two reagents in a biochip system, the method comprising steps of: (a) providing the biochip system having a biochip, a releasing actuator, a detector, a moving stage, and a processor, wherein the biochip comprises a plurality of patterned reagent wells with self-contained reagents that are connected to reaction wells via microchannels, the moving stage being mounted at the biochip, the moving stage being sized and configured to accurately position each of the reagent wells under the actuator; (b) releasing a first self-contained reagent from a first reagent well by positioning and activating the releasing actuator onto a first reagent well and transporting released first reagent to a first reaction well; (c) releasing a second self-contained reagent from a second reagent well by positioning and activating the releasing actuator onto a second reagent well and transporting released second reagent to the first reaction well; (d) moving the first reaction well to proximity of the detector using the moving stage; (e) measuring optical properties of fluid in the first reaction well using the detector; and (f) processing the optical properties of the fluid using the processor.

### BRIEF DESCRIPTION OF THE DRAWINGS

FIG. 1 shows a top view of a self-contained biochip with microfluidic channel connecting reagent cavities and reaction wells.

FIG. 2 shows an exploded top view of the three separate layers of the biochip, showing: (a) a reagent layer, (b) a microchannel layer, and (c) a reaction well layer.

FIG. 3 shows a cross section view of the biochip with microcap assembly and microfluidic channel, taken along line 3-3 in FIG. 1, showing the following sequence of operations: (a) before and (b) after the reagent being released from the reagent cavity and into microfluidic channels and reaction wells driven by a microactuator; the microcap assembly with a stopper and a pin being designed to reliably pierce the sealing thin film and open the cavity; and (c) the residual reagent in the reaction well being withdrawn via the waste port by a vacuum line.

FIG. 4 shows a section view of the self-contained biochip with a four-layer structure for dry reagent, showing the following sequence of operations: (a) the buffer solution and dry reagents being sealed in the separate cavities; (b) the first thin film being pierced, and the reagent buffer being moved into the dry reagent cavity and dissolves the dry reagent; and (c) the second thin film being pierced, and the reagent solution being released from the cavity into the microfluidic channels and reaction wells.

FIG. 5 shows the schematic diagrams of a biochip based analytical apparatus including a pressure microactuator, vacuum line, and optical detector.

FIG. 6 shows the schematic diagrams of a biochip situated on a rotational stage and the analytical apparatus including a pressure microactuator, vacuum line, and optical detector.

FIG. 7 shows the schematic diagrams of a biochip based analytical apparatus including a magnetic field generator.

FIG. 8 shows the schematic diagrams of a biochip based analytical apparatus including a heating and cooling element.

FIG. 9 shows an example of self-contained chip for chemiluminescence-based sandwich immunoassay protocol, showing the following states of the flow and reaction processes: (A) before and (B) after delivering the sample to the reaction wells; (C) washing away the unbound, and delivering the label conjugates; (D) washing away the unbound, and delivering the luminescent substrate.

FIG. 10 shows an mXP-CHIP (Microarray Expression Profiling Chip) microfluidic system for gene expression profiling.

FIG. 11 shows a laborious process of conventional microarrays (prior art).

FIG. 12 shows a schematic illustration of the processing steps for gene expression analysis using DNA microarrays.

FIG. 13 shows a comparison of differential expression of genes in human kidney and the universal human reference RNA (Stratagene).

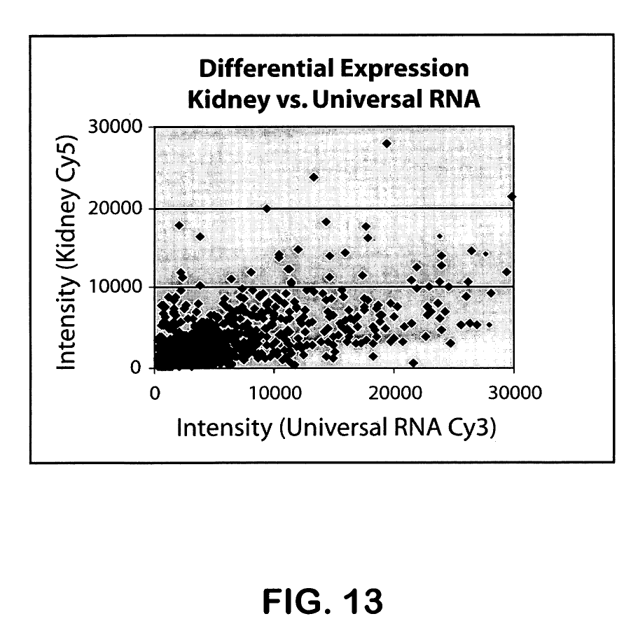

FIG. 14 shows a topical view of the mXP-CHIP device.

FIG. 15 shows the pressure-driven microfluidics for total automation.

FIG. 16 shows the microfluidic biochip for molecular diagnostics applications.

### DETAILED DESCRIPTION OF EMBODIMENTS OF THE INVENTION

This invention is described in various embodiments in the following description with reference to the figures. While this invention is described in terms of the best mode for achieving this invention's objectives, it will be appreciated by those skilled in the art that variations may be accomplished in view of these teachings without deviating from the spirit or scope of the invention. This description is made for the purpose of illustrating the general principles of the invention and should not be taken in a limiting sense. The scope of the invention is best understood and determined by reference to the appended claims.

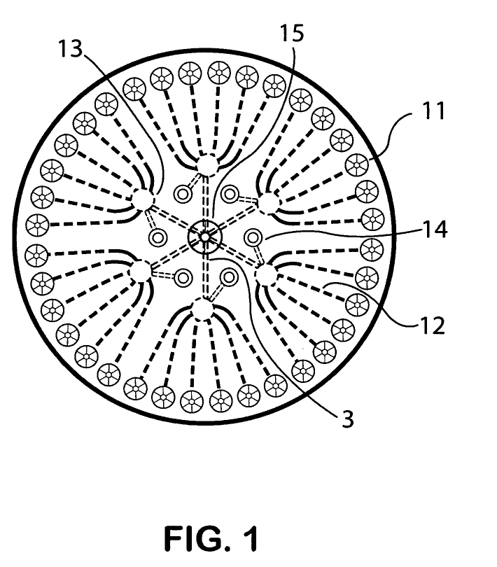

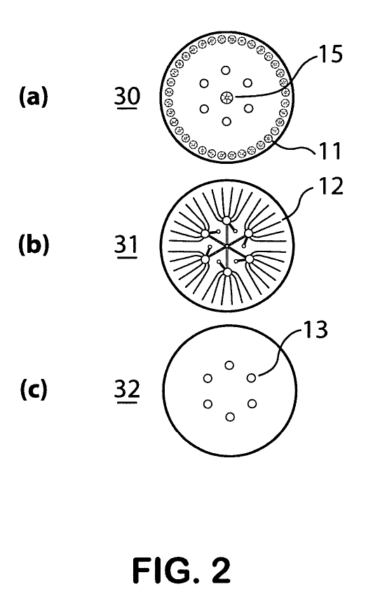

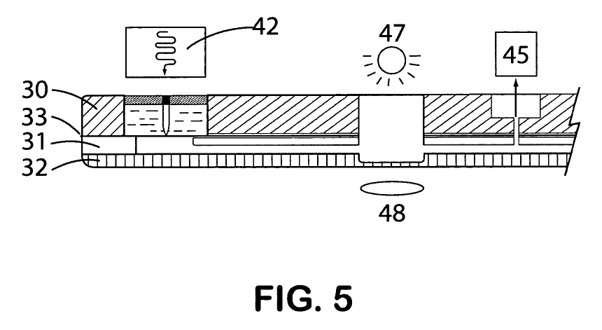

The pattern of the self-contained microfluidic biochip is designed according to the need of the assay and protocol. For example, the biochip (FIG. 1) consists of 6 sets of microfluidic pattern; it depends on the number of analyte and on-chip controls. Each set includes multiple (for example 6) reagent cavities 11, a reaction well 13, a waste port 14, and a network of microfluidic channel 12. The sample can be delivered into individual reaction wells directly or via a main sample port 15 for equal distribution to the reaction wells 13, for example under centrifugal forces by spinning the biochip in an analytical apparatus as discussed below in connection with FIGS. 5, 6, 7, and 8. The biochip body structure comprises a plurality of reagent cavities or wells and reaction wells via microchannels. Although the biochip has multiple sets of microfluidic patterns, each set of the pattern can be an independent sub-chip or pie-chip, which can be assembled into one circular biochip. This configuration offers a user's flexibility to customize his own multiple tests. In one embodiment, the chip has a three-layer composition: (shown in FIG. 2) (a) the top layer is a reagent layer 30, (b) the middle layer is a microchannel layer 31, and (c) the bottom layer is a reaction well layer 32.

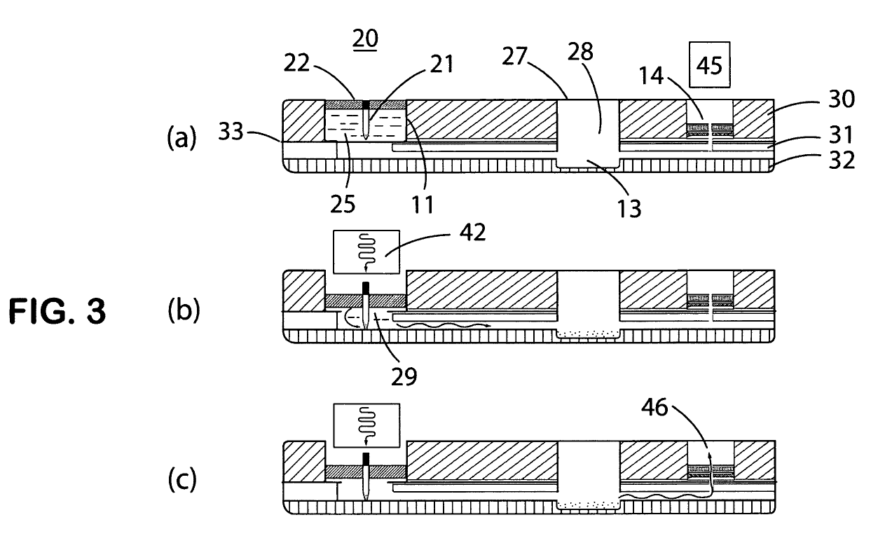

The reagent cavities 11 formed in the reagent layer 30 allow for the storage of various reagents or buffer solutions. The microchannel layer contains a network of microfluidic channels 36 are patterned on the bottom side of the layer. The microchannel layer and the reaction well layer form microfluidic channels, which connect the reagent cavities to reaction wells and to the waste port. The reaction well layer has a number of microwells, which are able to hold sufficient volume of samples or reagents for reactions. Reagent sealing means (shown in FIG. 3), which includes a thin film 33 located at the bottom of the reagent cavity and a microcap assembly 20 located at the top of the cavity, confines the reagent 25 in the reagent cavity. The thin film is breakable and is adhered to the reagent layer and the microchannel layer. The microchannel layer and reaction well layer are bonded by either chemical or physical methods. By way of illustrations, the various plastic layers may be bonded by applying ultrasonic energy, causing micro-welding at the adjoining interfaces.

The microfluidic biochip can be fabricated by soft lithography with polydimethyl siloxane (PDMS) or micro machining on plastic materials. PDMS-based chips, due to small lithographic depths, have volume limitations (<5 μl). When clinical reagents on the order of 5 μl to 500 μl, the layers are fabricated by micro machining plastic materials. The dimension of the reagent cavity could be easily scaled upward to hold sufficient volume of clinical samples or reagents. Soft lithography is best suited for microfabrication with a high density of microfluidic channels. But its softness properties and long-term stability remain a problem for clinical products. Therefore, the chip is preferably fabricated by micro machining on plastic materials. The linear dimension of a microfluidic channel is on the order of about 0.1 μm-2 mm, preferably between about 1 μm and 50 μm in cross section. In one embodiment, the plastic chips may be made of multi-layer polystyrene and polyacrylic. The biochip apparatus may further comprise a surface of the microfluidic channels that is treated with a surface tension reducing agent, such as fluorinated material, hydrophilic material, wetting agent or the like. In a preferred embodiment, the surface tension reducing agent does not interfere with the microfluidic operations. The cavity dimension of micro machining chips can be scaled up easily. It can be mass-produced by injection mold as a disposable chip.

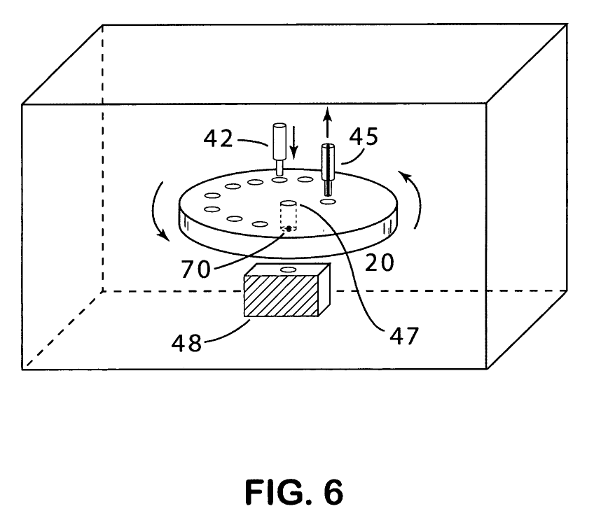

Referring also to FIG. 6, the biochip is placed on a rotational stage such as supported on a turntable (not shown) or on a spindle drive (not shown) connected to a motor (not shown), which positions a specific reagent cavity under a microactuator 42. All reagents are pre-sealed or pre-capped in reagent cavities. The microcap assembly is fabricated inside the reagent cavity to perform both capping and piercing. A pressure-driven microactuator controls the microfluidic kinetics. The microcap assembly has two plastic pieces: a pin 21 and a stopper 22. In the operation, the actuator engages with the assembly and pushes the element downward. The pin pierces through the thin film and opens the cavity. Then, the stopper is depressed downward to the bottom of the well. The stopper stays at the bottom of the well to prevent backflow. By this method, the microcap assembly opens the cavity as a valve 29 and let the reagent flow into the microfluidic channel. The configuration also prevents causing internal pressure build-up. The microactuator works like a plastic micro plunger or syringe, which is simple, rugged, and reliable. The movement of fluid is physically constrained to exit only through the microchannel and to the reaction well. A single actuator can manage a whole circle of reagent cavities.

After delivering the sample into the sample port or into one of the reaction well, the system sequentially delivers reagents one at a time into the reaction well and incubates for a predetermined time. The reaction well may be provided with a rubber cap 27 to prevent contamination by the environment, and the sample may be delivered directly into the reaction well by a probe piercing through the rubber cap 27, or via the sample port 15 at the center of the biochip. There is a large volume of air space 28 above the reaction well. With this design, air is allowed into the microfluidic system. No bubble is trapped in the microfluidic channel system. In practice, the actuator can also utilize the spare air in the reagent cavity to displace all of the residual liquid left in the microchannel into the reaction well, where there is plenty of air space. Therefore, the common problems associated with a microfluidic system, such as air bubbles, dead volumes, inhomogeneous distribution, and residual liquid left in the microfluidic channel, will not occur or affect the outcome of the test results. After the reaction, the residual reagent is removed away to an on-chip or off-chip waste reservoir. For example, a vacuum line 45 is situated atop the waste port 14 via a vented hole 46 to withdraw small volume of liquid from the reaction well.

The pre-loaded biochip is prepared and ready for use after shipment to the user. Therefore, the reagents, such as enzyme labeled antibody, should be stable for a long period (for example, 1 to 2 years or longer at room temperature). In their liquid form, many biological reagents are unstable, biologically and chemically active, temperature sensitive, or chemically reactive with one another. Because of these characteristics, the chemicals may have a short shelf life, may need to be refrigerated, or may degrade unless stabilized. Therefore some reagents are preferred to be stored in the dried form. One of dry reagent preparation methods is lyophilization, which has been used to stabilize many types of chemical components used in in-vitro diagnostics.

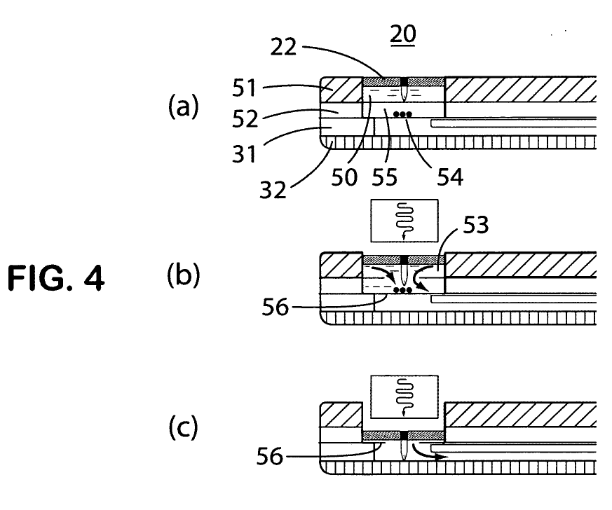

Lyophilization gives unstable chemical solutions a long shelf life when they are stored at room temperature. The process gives product excellent solubility characteristics, allowing for rapid liquid reconstitution. The lyophilization process involves five stages: liquid, frozen state, drying, dry, and seal. The technology allows lyophilized beads to be processed and packaged inside a variety of containers or cavities. In the case when dry reagents are involved, the biochip (shown in FIG. 4) has a four-layer composition: a reagent buffer layer 51, a dry reagent layer 52, a microchannel layer 31, and a reaction well layer 32. The reagent buffer layer with its patterned microwells allows for storage of the liquid form of reagents buffer 50 in individual wells. Buffer solutions are stable for a long period time.

The dry reagent layer contains dry reagent 54 in the dry reagent cavity 55 for rapid liquid reconstitution. When the actuator engages with the microcap assembly, it pushes the pin downward. The pin pierces through the first thin film 53, dissolves the dry reagent into buffer solution. Then the second thin film 56 is pierced, and the stopper is continuously depressed downward to the bottom of the cavity and forces the reagent mixture into the microchannel. Reactions take place in the reaction wells (not shown in FIG. 4), which are similar in structure to that shown in FIG. 3. The waste reagents may be removed by vacuum suction in a similar manner as in the previous embodiment. While FIG. 4 illustrates a particular embodiment in which a second, dry reagent is deployed, it is well within the scope and spirit of the present invention to deploy a second, wet reagent in place of the dry reagent. Further, it is contemplated that there could be a provision for more than two reagents, comprising a combination of dry and/or wet reagents.

While the embodiments are described in reference to one level of reaction using reagents delivered from multiple reagent cavities to a single reaction well, it is within the scope and spirit of the present invention that the biochip may be configured to perform two or more tiers of reactions in two or more reaction wells coupled in series by micro-channels. The reaction products from one or more reaction wells are fed into another reaction well (e.g., by pressurization using a plunger means (not shown) at the first reaction well or by centrifuging by spinning the biochip to cause the reaction products to move from one reaction well to another reaction well in series), where further reactions (i.e., a second tier of reactions) may take place using additional reagents from additional reagent reservoirs.

The analytical apparatus is designed to perform all necessary functions, such as reagent delivery, magnetic purification, mixing and incubation, heating and cooling, and optical detection on the microfluidic biochip. The analytical apparatus (as shown in FIGS. 5, 6, 7 and 8) is integrated with a moving stage, including a pressure-driven microactuator 42, a Peltier thermal cooler (heating and cooling element), an electromagnet, a vacuum line 45, a detector 48, and a microprocessor 72. The biochip may be supported on a turntable (not shown), or on a drive spindle (not shown) connected to a motor (not shown). Such details have been omitted from the schematic diagram in FIG. 6, so as not to obscure the present invention, but are well within the ability of a person in the art, given the present disclosure of the function and features of the present invention.

(A) The Moving Stage for Biochip Positioning:

The moving stage can accurately position any microwell on the biochip at a particular location to perform necessary function. The most convenient moving stage is a rotational stage. The rotational stage positions any microwell on the circular disc accurately for tasks of actuation, heating-cooling, magnetic purification, vacuuming, and detection, while an X-Y translation stage is suited for positioning the rectangular plate, like a 96-well microplate. In addition, the rotational stage also creates turbulence and mixing in the reaction well. Depending on the reactions, various rotational speeds can be controlled in order to create adequate mixing. The mixing process will enhance the biochemical reaction efficiency and thus reduce the incubation time. For a typical washing process, 20-30 seconds of mixing is sufficient.

(B) The Microactuator for Microfluidic Delivery:

The microactuator 42 is located above the disc set. After proper positioning of a specific microwell, the microactuator is used to create the downward pressure to push the pin and stopper element to break the seal and transport the fluid from the reagent well, into a microchannel, and then to the reaction well. The pressure-driven microactuator works as a plunger or a syringe to push the liquid out of the cavity. The microactuator is built with a motor that provides for linear motion. Linear screw provides continuous linear motion, while solenoid type actuator gives one-step motion. The microactuator will initiate fluid movement according to the sequence spelled out by the protocol and controlled by the microprocessor. The microactuator has a 5˜10 mm travel distance to press the microcap assembly to break the sealing film and push liquid into the microfluidic channel.

(C) Magnetic Field Generator for Purification and Filtering:

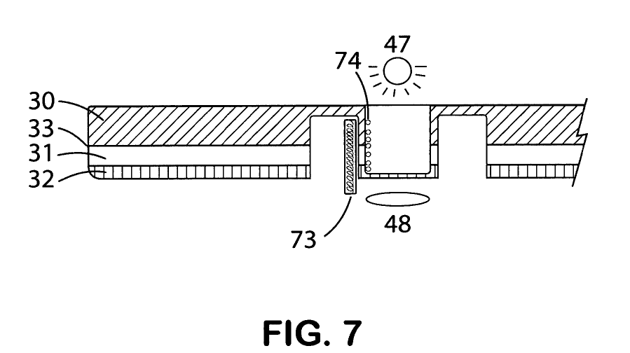

Magnetic beads 74 are super-paramagnetic. That is, they demonstrate magnetic properties when placed within a magnetic filed, but retain no residual magnetism when removed from the magnetic filed. This allows easy magnetic collection of microbeads and simple resuspension of the beads when the magnetic field is removed. Magnetic particles or microbeads have been immobilized with probe molecules for target capture. After the target capture, the magnetic particles remain in the reaction wells, while residual solution is washed away. Collection and resuspension of microbeads can be repeated simply and rapidly any number of times. This enables the buffer changes and extensive washing that will purify the captured target which is often required during molecular biology applications. The magnetic filed is generated either by moving a magnet in and out of place or by utilizing an electromagnet 73 (as shown in FIG. 7), which can be activated electronically.

(D) Heating and Cooling Element for Reaction Enhancement:

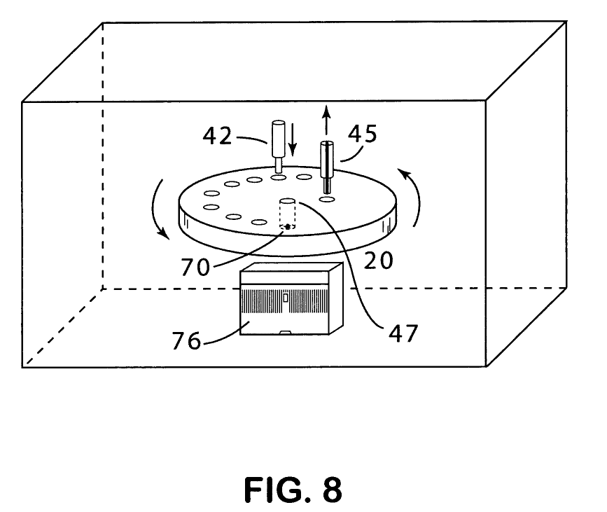

Molecular amplification processes, such as isothermal amplification or polymerase chain reactions (PCR), are performed at an elevated temperature. The typical temperature is ranged from 30-95° C. Since many biochemical reactions and amplification processes require cycling the temperature, the heating and cooling element should be able to raise and lower the temperature rapidly (about 2-5° C./per second) as shown in FIG. 8. A Peltier thermal heat pump 76 or a solid-state air/plate heat pump, is commonly used in the thermal cycler for both heating and cooling. In a Peltier pump, electric current is passed through the junction of two dissimilar electric conductors to produce or absorb heat, depending on the direction of the current through the junction. Standard cooling capacities range from about 20 W to more than 70 W with heating capacities in excess of 150 W. When a thermal heater is positioned beneath a reaction well, with a pocket configuration for thermal isolation, we have demonstrated that it can rapidly heat and cool the solution in the microwell.

(E) Vacuum Suction for Residual Solution Removal:

For multi-step reactions, each residual reactant needs to be removed for the next reaction. After the magnetic separation step as described above, the wash buffer needs to be aspirated. The residual reactant, wash buffer, or reaction waster can be transported to the waster storage on the chip or to an external waster storage. A variety of pumping mechanisms can be used to suck the liquid out of the reaction well. Diaphragm pump, vacuum pump, peristaltic pump, or air pump is a common choice to remove the liquid in the cavity. The microactuator 42 and vacuum line 45 may be actuated using linear actuators built with a motor-operated lead screw that provides for linear movement force.

(F) Optical Detection for Assay Measurement:

Chemiluminescence or bioluminescence, absorbance, fluorescence, are common optical methods for chemical and biological agent detection. Chemiluminescence or bioluminescence generates light through chemical reactions, no external light source is required for chemiluminescence or bioluminescence 70 detection. A photomultiplier tube (PMT) detector 48 can be used to measure the optical signal from the wavelength of 300-700 nm. Adding a photon counting electronic circuitry will significantly improve the performance and sensitivity. The detector has an internal current-to-voltage conversion circuit that is interfaced to a microprocessor. The integration time for each measurement is 100 ms and the dual kinetic assay can be easily performed. A single detector is sufficient to scan multiple reaction sites on the rotational stage. For certain applications, such as the enzyme-linked immunosorbent assay (ELISA) or fluorescence assay, a light source 47 can be implemented. However, other detection schemes may require a light source 47. The detector is one of the key elements that define the detection limit of the system. Depending on the sensitivity requirement, many detectors can be selected and used. Absorbance and fluorescence measurements require an external light source for illumination. Diode laser, LED, and lamp are typical light sources 47. The light source can be installed either above or below the biochip, and the transmission or reflection light can be detected with a detector. Photodiode, CCD, and PMT have different degree of sensitivity. The microprocessor will be responsible for controlling all the processes, mechanisms, timing, and fluid dynamics.

The photon counting photomultiplier tube has a very high amplification factor. This detector incorporates an internal current-to-voltage conversion circuit, and is interfaced with a microprocessor unit that controls the integration time. This detector has a very low dark count and low noise. The detector is packaged as part of a light tight compartment and is located either at the bottom or top of the transparent reaction well. One detector is sufficient to scan all reaction wells on the rotational stage. A collecting lens can be used to improve light collection efficiency. Arrangement of the reaction wells should minimize cross talk signals. A narrow band optical filter ensures detection of luminescence. The output of the detector is interfaced to a signal processor, which may be implemented within the apparatus shown in FIG. 6, or externally in a notebook computer or a digital meter. The optical signal corresponds to an analyte concentration, for example. Depending on the types of reactions undertaken, other types of detection schemes may be implemented without departing from the scope and spirit of the present invention. For example, electro-conductivity detection may be implemented using probes (not shown) inserted into the reaction mixture in the reaction well. The analytical apparatus may also include a probe (not shown) that can be positioned for injecting a sample into the sample port 15 on the biochip.

The control sequence for the various device components of the analytical apparatus may be configured in accordance with the desired reaction and reagent requirements. The control of components in a robotic analytical system is well known in the art. Accordingly, the disclosure of the present invention is enabled for one skilled in the art to configure the analytical apparatus in accordance with the function and features disclosed herein without undue experimentation.

The microfluidic biochip can be used for automating a variety of bioassay protocols, such as absorption, fluorescence, ELISA, enzyme immunoassay (EIA), light scattering, and chemiluminescence for testing a variety of analytes (proteins, nucleic acids, cells, receptors, and the like) tests. The biochip is configured and designed for whole blood, serum, plasma, urine, and other biological fluid applications. The assay protocol is similar to that manually executed 96-well microplates as described in U.S. Pat. No. 4,735,778. Depending on the probe used in reaction wells, the chips have the ability to react with analytes of interest in the media. The biochip is able to detect and identify multiple analytes or multiple samples in a very small quantity. The probes can be biological cells, proteins, antibodies, antigens, nucleic acids, enzymes, or other biological receptors. Antibodies are used to react with antigens.

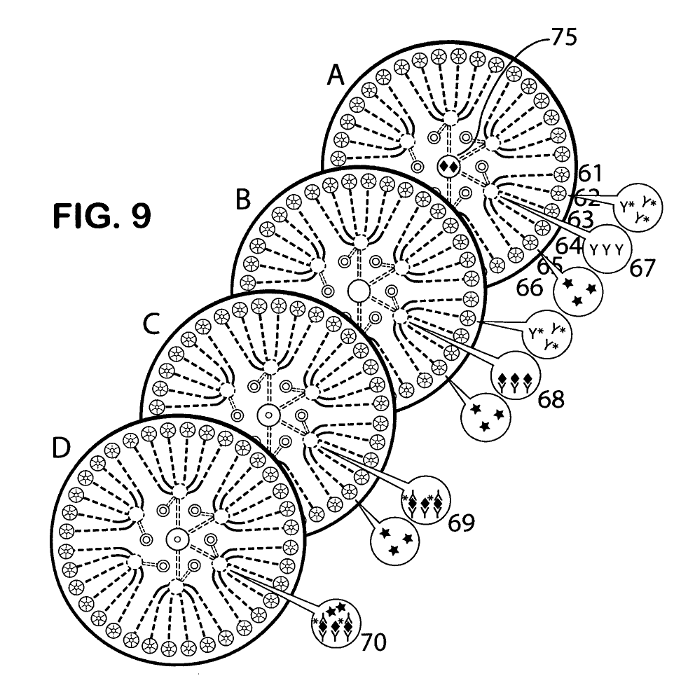

Oligonucleotides are used to react with the complementary strain of nucleic acid. By ways of illustration, for chemiluminescence-based sandwich immunoassay (FIG. 9), the reagent cavities are preloaded with pre-determined amounts of washing solutions 61, 63, 64, label conjugates 62, and luminescence substrate 65. The reaction well is immobilized with probes or capture molecules 67 on the bottom of the surface or on solid supports, such as latex beads or magnetic beads. There are many immobilization methods including physical and chemical attachments, which are well known to those who are skilled in the art. In one embodiment, once a sufficient sample 75 is delivered to the reaction well, the apparatus would automatically perform at least one of the following steps:
1. Let the sample or target incubate in the reaction well for approximately 5-10 minutes to form probe-target complex 68, followed by activating the vacuum line to remove the residual sample to the waste reservoir.
2. Dispense washing solution from a reagent cavity to the reaction well, followed by removing the unattached analyte or residual sample from the reaction well to the waste reservoir.
3. Move the label conjugate from the reagent cavity to the reaction well and incubate it, followed by removing the unattached conjugate to the waste reservoir.
4. Wash the reaction sites two or three times with washing solutions from reagent cavities to remove unbound conjugates, followed by removing the unattached conjugate to the waste reservoir.
5. Deliver chemiluminescence substrate solution 64 to the reaction well.
6. The reaction site would start to emit light only if the probe-target-label conjugate complex 69 is formed. The signal intensity is recorded. The detector scans each reaction well with an integration time of about one second per reading.

Chemiluminescence occurs only when the sandwich immuno-complex 69 ((e.g. Ab-Ag-Ab*), positive identification) is formed. The labeled enzyme amplifies the substrate reaction to generate bright luminescence 70 for highly sensitive detection and identification.

Microarray Genomic Profiling

Some aspects of the present invention relate to rapid and automated Microarray Expression Profiling Chip (mXP-CHIP), system, and methods for gene expression profiling. Microarray genomic profiling is a powerful tool in molecular characterization of diseases, but remained restricted to research, having not yet made a breakthrough as a diagnostic tool in disease identification and drug development. This is due to current time and labor consuming macro-scale platforms and the inconsistencies in generating results. In one embodiment, the mXP-CHIP system provides a miniaturized and automated system to integrate all steps of probe synthesis, labeling, and hybridization, configured and adapted for reducing the risk of contamination to a minimum and significantly decreasing assay costs due to the consumption of lower reagent volumes and shorter assay times. Microarray assays would then be performed with higher confidence and cost-efficiency for the screening of target genes in the academic research of genetic, immune and infectious diseases, and for relevant drug target identification and validation in the pharmaceutical industry.

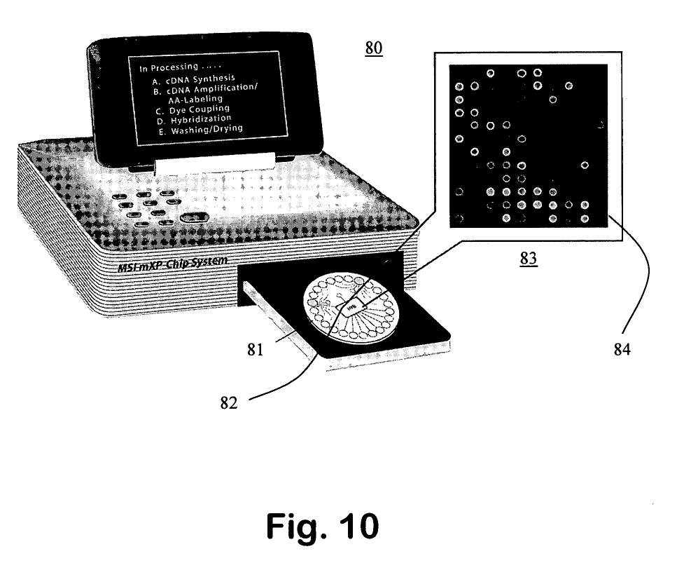

Microfluidic technology, by nature, shows excellent advantages for integrating the multiple steps of the DNA microarray process. FIG. 10 shows an mXP-CHIP microfluidic system for gene expression profiling, that utilizes parallel processing, microfabrication and microfluidic technologies in order to provide biochemistry automation in an about 3″ compact disc package. In one embodiment, the integrated probe generation and hybridization within microarrays may be up to 30,000 spots into one single sterilized device. The need for a network of tubing connected to external reservoirs and pumps of a prior system is eliminated. The mXP-CHIP system utilizes a simple pressure driven microactuator, vacuum line, and heating elements in order to control fluid dynamics and regulate biochemical reactions configured and adapted for reducing multistep reactions to a single step. The mXP-CHIP and system integrates all the steps of probe synthesis, labeling and purification, probe hybridization to a microarray, post-hybridization washes and drying so that the array is ready for scanning.

FIG. 10 shows a system 80 comprising a miniature compact, automated, self-contained microarray expression profiling chip 81 (mXP-CHIP) for automated inline RNA probe processing from synthesis to hybridization. The mXP-CHIP is equipped with a centered hybridization well 82 (HYB) that contains a whole human genome microarray 83 of DNA spots (20,000-30,000 genes). The system integrates all the required reagents in the surrounding microwells and the steps of probe synthesis, labeling, post-labeling purification, dual-color hybridization (magnified section 84 of the array in the right upper comer), and post-hybridization processing to the point that the arrays would be ready for scanning. The mXP-Chip utilizes parallel processing, microfabrication and integrated microfluidic technologies to provide biochemistry automation in a small disc plastics package.

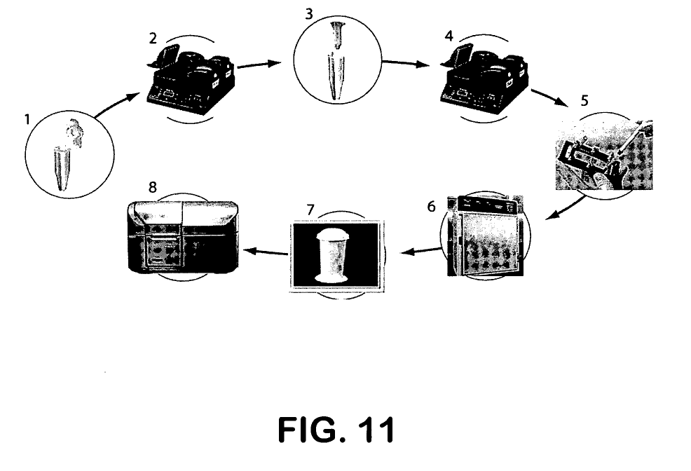

FIG. 11 shows a prior art illustration of a laborious process of conventional microarrays. The prior art microarrays may include steps of: labeled probe is generated by (step 1) mixing the reaction components together in a tube and (step 2) incubating in a programmable thermocycler; (step 3) after cRNA synthesis and labeling, the solution is transferred to a silicagel-column, which purifies labeled cRNA from enzymes and unincorporated dye molecules via centrifugation; (step 4) the collected purified cRNA is then heat-fragmented in the thermocycler and (step 5) then applied to the array in the hybridization chamber; (step 6) hybridization takes place in an oven at about 60° C. under gentle rotation; (step 7) post-hybridization washes are performed consecutively in 2-3 jars with washing solution of increasing stringency, including transfer of the slides between the jars; and (step 8) the slides are then blow-dried with a nitrogen gun or spin-dried in a centrifuge before being scanned.

Many firms and research institutes are actively engaged in developing microfluidic technologies. Currently there are commercialized stations (for example, Genomic Solutions, by Perkin Elmer, Boston, Mass. and Gene Chips™ by Affymetrix, Santa Clara, Calif.) that only perform hybridization/washing. These devices are expensive to procure & maintain, and do not integrate the important steps of probe generation (i.e. cRNA synthesis and labeling as well as cRNA purification and array hybridization) into the automated process. Thus gene expression profiling remains laborious and susceptible to inconsistent manual operations with these commercial stations.

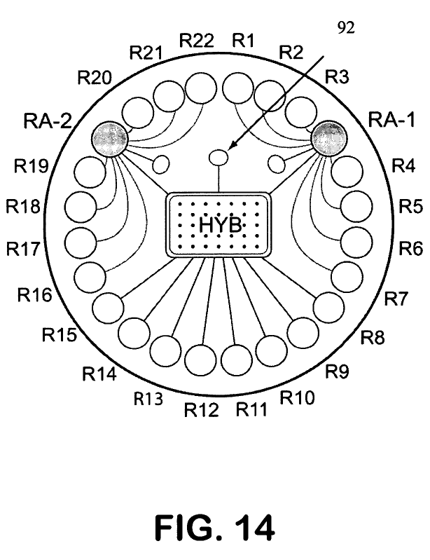

During the mXP-CHIP assay procedure, the actuator sequentially aliquots a pocket of solution from one microwell to another, initiates enzymatic and non-enzymatic chemical reactions, rinses off unbound reagents, and removes excess solution into a waste reservoir with the assistance of the vacuum line. FIG. 14 shows a topical view of the mXP-CHIP, wherein the mXP-CHIP's unique design is not affected by common microfluidic problems, such as dead volumes, air bubbles, or residual liquid inside the microfluidic system. These problems have hampered the technology from becoming commercially viable for clinical diagnostics in the past. The application of a geometrically defined system, in combination with defined input volumes, decreases reagent handling variations within the steps of probe generation and hybridization. Even more importantly, this system is sized and configured for gene expression profiling, whereby the chip can process multiple probes at the same time and subsequently perform dual-color hybridization.

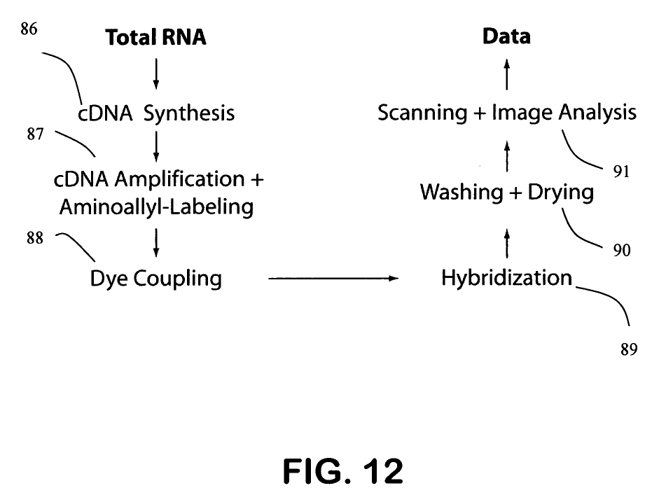

FIG. 12 shows a schematic illustration of the processing steps for gene expression analysis using DNA microarrays. The procedure starts from minute amounts of extracted and purified total RNA (5-10 ng) as sources for probe generation, i.e. cDNA synthesis 86, followed by cDNA amplification/aminoallyl labeling 87, fluorescent dye coupling 88, hybridization 89 to an array of DNA target elements, subsequent washing 90 of unbound material, drying of the array & scanning 91 and data acquisition by image analysis.

### EXAMPLE NO. 1

The system as shown in FIG. 10 is designed and configured to simultaneously synthesize and label probes from two samples and hybridize the two probes to an integrated array of 400 spotted DNA elements. These elements would represent 400 well-characterized unique genes and are selected so that according to preliminary data (FIG. 10), 2/3 of the genes are known to be highly expressed between normal human kidney (Asterand, Detroit, Mich.) and the Universal Human Reference RNA™ (Stratagene, La Jolla, Calif.). While 1/3 are known to show a low to no differential expression (between 0.5- and 1.5-fold) between the two specimens. The expression profiles have been generated using the Agilent Human 1A Oligo Microarray™ (22K array) and the Agilent platform for probe synthesis, hybridization and scanning. The slight bias of the data points towards the Reference RNA is caused by the fact that the Universal RNA is a pool of total RNAs of 10 different human tissues. Thus for the majority of the genes displayed, the sum of gene expression in the pool is higher than in kidney alone.

In one embodiment, gene expression profiling using the mXP-CHIP system is comprised of three major processing steps: 1) cDNA synthesis, 2) fluorescent dye coupling, and 3) hybridization. The whole process starts with probe synthesis from 1-5 ng of purified total RNA from normal human kidney (Asterand) and the Universal Human Reference RNA™ (Stratagene). The probe generation starts with (A) a reverse transcription from minute amounts of total RNA (5-10 ng) using a chimeric RNA/DNA-primer (using the Ovation Nanosample Amplification™ kit of Nugen (San Carlos, Calif.)), (B) the formation of double-stranded cDNA, and (C) an isothermal step, in which cDNA is linearly amplified while incorporating aminoallyl-dUTP. The rapid accumulation of cDNA (5-10 μg) is the result of a cascade of subsequent reactions taking place: primer annealing, cDNA strand synthesis by DNA polymerase, strand displacement and RNA cleavage of the chimeric primer by RNase H. The aminoallyl-labeled cDNA can then be coupled to amine-reactive Cy3 or Cy5 dyes for fluorescent labeling in a following step (not shown).

### EXAMPLE NO. 2

cDNA Synthesis & Amplification: cDNA synthesis includes three different reaction steps: (A) a reverse transcription (RT) step; (B) the formation of double-stranded cDNA step; and (C) the isothermal amplification (IA) step. In the RT, the mRNA (poly A+) portion within the total RNA is transcribed into single-stranded cDNA using a chimeric RNA-DNA primer. The primer binds with its DNA part to the poly(A) tail of the mRNA but the RNA part stays single-stranded. In this way the resulting RNA-cDNA complex has a unique RNA target sequence at the 5′ end of the cDNA. After fragmentation of the RNA in the RNA-cDNA complex, DNA polymerase is used to synthesize a second strand including DNA complementary to the unique 5′ RNA sequence of the first strand. The result is a double-stranded cDNA with a unique RNA/DNA heteroduplex at the 5′ end. In the IA part of the unique 5′ RNA sequence is removed by added RNase H. The exposed cDNA sequence is then available for binding a second chimeric RNA-DNA primer. The DNA polymerase subsequently extends the strand while incorporating aminoally-dUTP into the de novo synthesized sequence starting at the 3′ end of the primer and displacing the existing forward strand. The process of chimeric primer binding, DNA synthesis, strand displacement, and RNA cleavage of the primer is repeated multiple times, resulting in a rapid accumulation of single-stranded aminoallyl labeled cDNA, that is complementary to the original mRNA (antisense cDNA). The cDNA is stopped by heat-inactivation and the product, i.e. aminoallyl-labeled cDNA, is purified from enzymes and unincorporated nucleotides with magnetic beads (Dynal Biotech, Lake Success, N.Y.).

### EXAMPLE NO. 3

Fluorescent Dye Coupling: The fluorescence labeling of the cDNA is achieved in a simple chemical by coupling of NHS-ester cyanine 3 (Cy3) or cyanine 5 (Cy5) to aminoallyl groups of the cDNA. The uncoupled dye is removed by purification with magnetic beads process (Dynal).

### EXAMPLE NO. 4

Hybridization: The two differently labeled cDNAs from sample A and B are pooled together with competitor DNA & control RNA and subjected to dual-color hybridization onto the integrated array of 400 printed DNA elements. Hybridization would be performed for 16 hours following the protocols for conventional hybridization with microarrays. The hybridized chip would be subjected to post-hybridization washes and scanning for image analysis and the collection of gene expression data.

In co-pending applications, U.S. Ser. No. 10/338,451 and PCT WO2004/062804, entire contents of which are incorporated herein by reference, disclose certain microfluidic chip platform for configuring the mXP-CHIP structure. The principle behind mXP-CHIP operations is that the system sequentially delivers reagents one at a time from a reagent well to a reaction well, and then to the hybridization well. The mXP-CHIP has a 3D structure that makes it easier to transfer the fluid to different locations. A pressure driven microactuator (external) is used to initiate fluid transport from well to well, while a vacuum port 92 and vacuum line (external) with a vent hole is associated with each reaction well for fluid removal. Depending on the assay protocol and the number of samples, the mXP-CHIP or cartridge can be fabricated with an array of patterned microchannels and microwells. FIG. 14 shows a topical view of the mXP-CHIP. The diagram shows the chip with sets of microwells for reagents (R1-R22), reactions like probe synthesis/labeling (RA-1 & RA-2), and hybridization 82 (HYB).

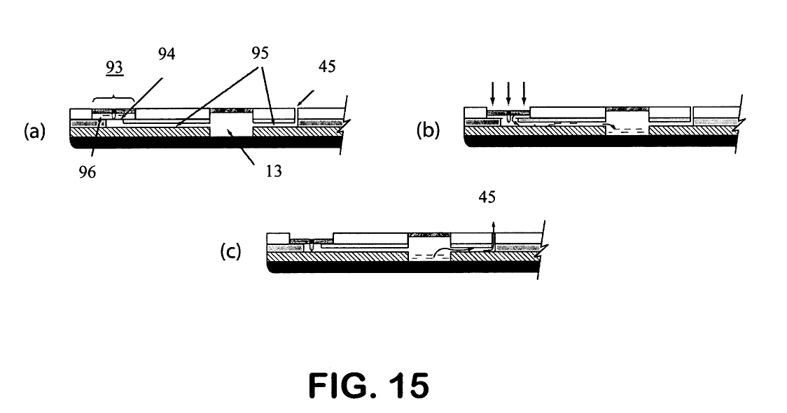

In one embodiment, the chip 81 has a circular format (d=3 inch). It sits on a rotational stage for accurate microwell positioning. FIG. 15 shows the pressure-driven microfluidics for total automation. The stopper element 93, with a stopper 22 and a pin 21, is designed to reliably pierce the thin film gasket 94 and open the microwell 96. The sequence of microfluidics is as follows: (FIG. 15a) the reagent solution is sealed in the separate cavity of the microwell; (FIG. 15b) the reagent solution is released from the microwell into the microchannels 95, and reaction wells 13; and (FIG. 15c) the reactive solution in the reaction well is withdrawn to the external waste reservoir by a vacuum line 45. Repeat the above processes to complete a sequence of reactions. In one embodiment, the mXP-CHIP consists of a three-layer microwell structure (as shown in FIG. 15): a reagent well layer 30 (top), a microchannel layer 31 and a reaction well layer/hybridization well (bottom) layer. The reagent microwells have a pattern allowing for the storage of reagent solutions. Each microwell has a sufficient amount of liquid for a reaction (up to 500 μl). Between layers, there are gasket thin films. These gaskets not only separate the layers, but also contain a network of microfluidic channels (100 μm) patterned on the bottom side of the gasket.

These microfluidic channels connect the reagent wells to the reaction wells and to the hybridization well or waste port. In some embodiments, two types of thin film gaskets are used, one made of PDMS and one from a polyacrylic composite. In one embodiment, the fluidic chip bodies are made from plastic materials (e.g. polystyrene, polyacrylic, etc.), and can be mass-produced by injection molding. The microactuator works like a plastic micro syringe, is simple, and reliable. It is operated by a micro stopper element that can be plugged inside the microwell—capping the reagent in a microwell and piercing the gasket at the bottom of the well. FIG. 15 shows the micro stopper element with two partially connected plastic pieces (a stopper and a pin).

When the external actuator, similar to a linear screwdriver, engages with the micro stopper element, it pushes the pin element (right on top of the film) downward without causing internal pressure build-up. The pin pierces through the thin film gasket, and stops at the bottom of the microchannel. The downward force separates the plastic pieces and the stopper is depressed downward to the bottom of the well. The stopper stays at the bottom of the well to prevent backflow. By this method the stopper element opens the microwell as a one-way valve. The movement of fluid is physically constrained to exit through a microchannel to the reaction well. To move all the residual liquid (1-3 μl) left in the microchannel into the reaction well, spare airs in the reagent wells are intentionally provided to displace all of the fluid left in the microchannel.

A reaction/hybridization well, capped with a rubber seal with a small vent hole, has a large liquid and air volume. With this design, air is allowed into the microfluidic system. Therefore, the common problems associated with microfluidic systems, such as air bubbles, trapped air, dead volumes, inhomogeneous distribution, and backflow into the channels, will not affect the outcome. Furthermore, the microwell/rubber seal structure provides fluid accessibility. Therefore, every step of reaction in the protocol can be monitored for yields and qualities. A vacuum line, with a port, is associated with each reaction and hybridization well to provide fluid removal from each reaction well to a waste storage and transport fluid from the reaction wells to the hybridization wells. The make-up air from the vent hole would replace the volume of fluid been removed. The hybridization slide (about 1 cm×1 cm) can be placed in or taken away from a hybridization well for laser scanning.

### EXAMPLE NO. 5

The bioassay process includes four major steps: simultaneous cDNA synthesis; cDNA linear amplification & aminoallyl labeling from two samples (as described in Example No. 1); fluorescent dye coupling of the cDNAs; and dual-color hybridization to the integrated array of spotted DNA elements (as shown in FIG. 12). Both samples are purified, once after aminoallyl and once after fluorescent dye coupling, with magnetic beads (Dynal Biotech, Lake Success, N.Y.), pooled together with Competitor DNA (for specific hybridization) and Control RNA (for signal normalization) and applied to a microarray for hybridization. In the first step of the process the mixture of total RNA and chimeric RNA-DNA primer is manually loaded into the reaction wells RA-1 and RA-2. The rest of the steps are performed in an automated fashion (FIG. 12).

### EXAMPLE NO. 6

cDNA Synthesis:
1. Load a 7 μl mixture of total RNA from sample A and First Strand Primer Mix as well as a 7 μl mixture of sample B and First Strand Primer Mix into RA-1 and RA-2, respectively. Incubate at 65° C. for 5 minutes (denaturing step).
2. Cool RA-1 & RA-2 to 48° C. and forward 13 μl of First Strand Master Mix from reagent wells R1 and R22 to RA-1 and RA-2, respectively. Mix by rotating disc back and forth for 1 minute and incubate for 1 hour at 48° C. (cDNA first strand synthesis).
3. Cool RA-1 & RA-2 to 37° C. and forward 20 μl of Second Strand Master Mix from R2 and R21 to RA-1 and RA-2, respectively. Mix by rotating disc back and forth for 1 minute and incubate at 37° C. for 30 minutes (CDNA second strand synthesis).

### EXAMPLE NO. 7

Isothermal PCR/Aminoallyl-Labeling
1. Forward 120 μl of SPIA™ Master Mix from R3 and R20 to RA-1 and RA-2, respectively. Mix by rotating disc back and forth for 1 minute. Heat RA-1 and RA-2 to 50° C. and incubate for 90 minutes.
2. Heat RA-1 and RA-2 to 95° C. and incubate for 5 minutes (enzyme inactivation).
3. Cool reaction the chamber to 22° C. and forward the magnetic bead solution from R4 and R19 to RA-1 and RA-2, respectively. Incubate at room temperature for 5 minutes (cDNA is captured by the beads).
4. Immobilize the magnetic beads with an external magnet and vacuum the reactant into the waste/vacuum line. Forward 100 μl wash buffer from R5 and R18 to RA-1 and RA-2, respectively. Incubate for 1 minute and then remove wash buffer.

### EXAMPLE NO. 8

Fluorescent Dye Coupling:
1. Forward 10 μl of Cy3 Dye Mix and Cy5 Dye Mix from R6 and R17 to RA-1 and RA-2, respectively. Incubate for 60 minutes at 22° C.
2. Forward 100 μl of wash Buffer from R7 and R16 to RA-1 & RA-2, respectively and incubate 1 minute. Hold the magnetic beads at the bottom of RA-1 and RA-2 and remove wash buffer.
3. Forward 50 μl elution buffer from R8 and R15 to RA-1 and RA-2, respectively. Incubate for 5 minutes and move the reactant (including the purified cDNA) from RA-1 and RA-2 into the hybridization chamber HYB (containing the microarray).

### EXAMPLE NO. 9

Hybridization & Post-Hybridization Processing:
1. Forward 50 μl of Hybridization Mix from R9 to HYB and rotate for 30 seconds. Heat HYB to 95° C. and incubate for 3 minutes. Cool HYB to 60° C. and incubate for 16 hours.
2. Replace the hybridization solution with 100 μl of Wash Buffer A from R10. Cool the array chamber down to 25° C. and incubate for 3 minutes. Repeated washes with another 100 μl of Wash Buffer A (R11), Buffer B (R12), and then Buffer C (R14), and for 3 minutes while rotating the disc at 25° C. The microarray would be dried and subject to scanning.

Microarrays are critical in the process of determining the function of the human genome, speeding up the discovery process many times over. Microarray biochips in combination with microfluidics are revolutionizing drug discovery, laboratory testing and device development leading to a new paradigm for the big pharmaceutical companies. Point of care diagnostics is poised to make a leap forward as microarrays and microfluidics chips become less expensive. Home diagnosis with disposable chips may become a reality within 10 years. The worldwide market for microarrays and microfluidics is expected to grow to $1 Billion by 2005. This expected growth would mean an annual growth rate of 21.8% during the 5-year forecast period since 2000 (Business Communications Co., Inc).

The chip system is not restricted to serve as a reliable tool for gene expression profiling. The chip design and the control software can be flexibly modified to also address any application that involves nucleic acid probe labeling and array/target hybridization, including different types of genotyping, such as comparative genomic hybridization (CGH), detection of single nucleotide polymorphisms (SNPs) or identification of thread agents like bacteria and viruses. The self-contained design of the chip makes the device especially suitable as a portable system for out-lab use in remote areas. Microarrays in combination with microfluidics have the potential to also significantly decrease assay costs due to the consumption of lower reagent volumes and shorter assay times. Thereby microarray assays can be used with much higher confidence and cost-efficiency for: (1) screening of candidate genes in the research of genetic, immune & infectious diseases; (2) drug target identification and validation by the pharmaceutical industry, and (3) identification of threat agents (bacteria and viruses).

Molecular Diagnostics

The microfluidic biochip and apparatus is used to automatically carry out all target (pathogen ribosomal RNA=rRNA) capture and processing steps necessary for measuring the pathogens' loads in specimens. For this purpose, the biochip is patterned with microwells and a network of microchannels for fluid transport. In one example, the microwells include 18 reagent wells; three reaction wells for simultaneous capture, amplification, and detection of one specimen; and the two control ribosomal RNAs (rRNAs) for Chlamydia trachomatis (CT) and Neisseria gonorrhoeae (GC). All necessary reagents are packaged in sufficient quantities within sealed reagent wells. The compact analytical apparatus utilizes innovative microfluidic technology for specific target capture on beads, target purification form of other biomaterial in the specimen, transcription-mediated amplification (TMA) of the target, and luminescent detection of both pathogens using dual kinetic assay (DKA). The system integrates the steps of: reagent delivery, reagent mixing, reagent heating and cooling, and magnetic separation in a programmable walk-away fashion.

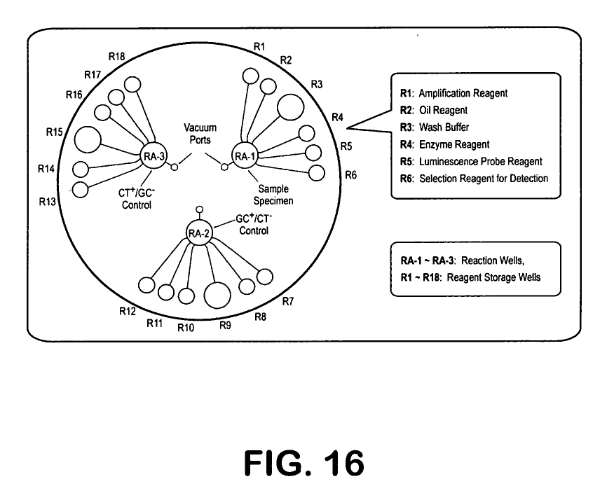

FIG. 16 shows the topical view of the microfluidic biochip of the invention. The diagram shows the chip with three groups of microwells: each group is comprised of 6 microwells sets at two different sizes for reagent storage (R), and one reaction well (RA) for probe processing. The legend on the left shows the set of reagents sequentially forwarded from the reagent wells to each RA within a group for probe processing (pathogen rRNA capture, rRNA amplification and detection). The RAs are connected to individual vacuum lines for liquid aspiration. The chip is designed to simultaneously capture, amplify and detect probes from a patient sample and the two controls for C. trachomatis and N. gonorrhoeae. The bottom of the reaction wells is transparent, and the luminescence in each reaction well is measured by rotating the chip into a precise position above the optical detector.

The bioassay process involves generating (1) capture of pathogen rRNA targets by magnetic beads (coated with capture oligonucleotides), (2) transcription mediated amplification of captured target(s), and (3) target detection via a chemiluminescent dual kinetic assay. The steps are performed in an automated fashion given below.

A. Capture of Target rRNA:
1. Incubate the specimen capture-reagent mixture in RA-1 to RA-3 at 62±1° C. for 30±5 min.
2. Agitate the mixture by high-frequency alternate orbital rotation for 1 min, and incubate the mixture at room temperature for 30±5 min.
3. Activate the electromagnet and wait for 5-10 min for magnetic beads to be captured. Then vacuum the supernatant into the waste line.
4. Forward 1 ml of APTIMA Wash Buffer from R3, R9 and R15 into RA-1 to RA-3, respectively, and resuspend beads (magnet off) by orbital rotation for 1 min; reactivate magnet for 5-10 min. Then aspirate the supernatant into the waste line.

B. Transcription Mediated Amplification:
1. Forward 75 μl of the amplification reagent from R1, R7, and R13 into RA-1 to RA-3, respectively; mix by rotation; then forward 200 μl of oil reagent from R2, R8, and R14 into RA-1 to RA-3, respectively; and agitate by high-frequency alternate rotation for 1 min. Incubate the mixture at 62±1° C. for 10±5 min. Then incubate at 42±1° C. for 5±2 min.
2. Forward 25 μl of the enzyme reagent from R4, R10, and R16 to RA-1 to RA-3, respectively, and gently mix by rotation. Incubate mixture at 42±1° C. for 60±15 min.

C. Dual Kinetic Assay for Detection:

C1. Hybridization
1. Forward 100 μl of the probe reagent from R5, R11, and R17 into RA-1 to RA-3, respectively, and agitate by high-frequency alternate rotation for 30 sec. Incubate the mixture at 62±1° C. for 20±5 min. Then incubate at room temperature for 5±1 min.

C2. Selection
2. Forward 250 μl of the selection reagent from R6, R12, and R18 into RA-1 to RA-3, respectively, and agitate by high-frequency alternate rotation for 10 sec. Incubate the mixture at 62±1° C. for 10±1 min.

C3. Detection
3. Decrease the reagent temperature to room temperature (22-25° C.) and incubate for 15±3 min.
4. Record chemiluminescence as relative light units by the detector.

Nucleic Acid Extraction

The microfluidic biochip platform is also useful for both the concentration and purification of nucleic acid targets from whole blood to enable nucleic acid amplification. The procedure for isolating DNA requires, in general, four important steps: effective disruption of cells and organisms, capture of DNA onto the magnetic beads, repeat washing and purification of DNA from inhibitors and other cell components, and elution of pure DNA in small amounts of buffer. The biochip is pre-loaded with all the necessary reagents and washing buffers. The dimension of the microwells is constructed with sufficient volume to manage the required solutions. The fully-automated DNA-extraction assay protocol includes Step 1. Cell disruption and protein digestion to release DNA; Step 2. DNA concentration by adsorption to the surface of magnetic beads; Step 3. Magnetic separation of the intact DNA-bead complex and extensive washing to remove inhibitors and cellular debris; and Step 4. Elute purified DNA from the magnetic beads will be removed by a multichannel pipetter for downstream PCR application.

Some aspects of the invention is to provide an analytical apparatus or a biochip system for use with a microfluidic biochip that comprises a plurality of patterned reagent wells, each reagent well having a self-contained reagent, the reagent wells being connected to their respective reaction wells via certain respective microchannels. In one embodiment, at least one of self-contained reagents comprises magnetic particles.

In one embodiment, the analytical apparatus or the biochip system comprises a micro mechanical actuator for delivering downward pressure to transport at least one of the reagents from the respective patterned reagent wells into a first reaction well, the target reagent well being moved by the moving stage to be at proximity of the actuator.

In one embodiment, the analytical apparatus further comprises a detector for measuring optical properties of the fluid combining at least two reagents in the first reaction well, the target reaction well being moved by the moving stage to be at proximity of the detector.

In one embodiment, the analytical apparatus further comprises a moving stage mounted at the biochip, wherein the moving stage is sized and configured to accurately position each of the reagent wells under the actuator, position the first reaction well above or below the detector, and position the first reaction well above a heating and cooling element. In a further embodiment, the moving stage is an X-Y translation stage or a rotational stage.

In one embodiment, the analytical apparatus further comprises a heating and cooling element for controlling temperature of the fluid within the reaction well. In a further embodiment, the heating and cooling element is a Peltier thermal heat pump.

In one embodiment, the analytical apparatus further comprises a processor configured to control the moving stage and process the optical properties, wherein the optical properties are chemiluminescence or bioluminescence signals.

In one embodiment, the analytical apparatus further comprises a vacuum suction connected to the first reaction well, wherein the vacuum suction is configured for removing the fluid away from the first reaction well.

In one embodiment, the analytical apparatus further comprises a magnetic field generator situated in proximity of the first reaction well, wherein the generator has on and off switching mechanisms, the magnetic field generator being an external magnet or a built-in electromagnetic element.

In one embodiment, the analytical apparatus further comprises a light source located above or below the biochip, wherein the light source illuminates the first reaction well for light absorbance or fluorescence measurement.

Some aspects of the invention is to provide a method for measuring optical properties of a fluid combining at least two reagents in a biochip system, the method comprising some of the following steps: providing the biochip system having a biochip, a releasing actuator, a detector, a moving stage, and a processor, wherein the biochip comprises a plurality of patterned reagent wells with self-contained reagents that are connected to reaction wells via microchannels, the moving stage being mounted at the biochip, the moving stage being sized and configured to accurately position each of the reagent wells under the actuator; releasing a first self-contained reagent from a first reagent well by positioning and activating the releasing actuator onto a first reagent well and transporting released first reagent to a first reaction well; releasing a second self-contained reagent from a second reagent well by positioning and activating the releasing actuator onto a second reagent well and transporting released second reagent to the first reaction well; moving the first reaction well to proximity of the detector using the moving stage; measuring optical properties of fluid in the first reaction well using the detector; and processing the optical properties of the fluid using the processor.

While the present invention has been particularly shown and described with reference to the preferred embodiments, it will be understood by those skilled in the art that various changes in form and detail may be made without departing from the spirit, scope, and teaching of the invention. For example, while the present invention has been described in reference to a biochip having circular array of reagent wells and reaction wells, the present invention can well be implemented in a biochip having a rectangular array, or an array of other geometries. Furthermore, the present invention may be implemented on a biochip having a footprint or format compatible to a 96-well micro-titer plate, so that compatible apparatus may be used to handle the biochip, such as laboratory robotic equipments. Still further, while the invention has been described in reference to a process using a biochip analytical apparatus that includes a detector, the present invention may be implemented in a process using an apparatus that allows the reactions to complete in the biochip, and then the biochip is transferred to another apparatus that is dedicated to detection of the final reaction product.

The biochip system and methods of the present invention are not restricted to serve as a reliable tool for gene expression profiling, molecular diagnostics, and nucleic acid extraction. The chip design and the control software can be flexibly modified to also address any application that involves nucleic acid probe labeling and array/target hybridization, including different types of genotyping, such as comparative genomic hybridization (CGH), detection of single nucleotide polymorphisms (SNPs) or identification of thread agents like bacteria and viruses. The self-contained design of the chip makes the device especially suitable as a portable system for out-lab use in remote areas. Accordingly, the disclosed embodiments are to be considered merely as illustrative and the present invention is limited in scope only as specified in the appended claims.

1. An analytical apparatus for use with a microfluidic biochip that comprises a plurality of patterned reagent wells with self-contained reagents that are connected to reaction wells via microchannels, the apparatus comprising:
(a) a micro mechanical actuator for delivering downward pressure to transport at least one of said reagents from said patterned reagent wells into a first reaction well;
(b) a detector for measuring optical properties of fluid in said first reaction well;
(c) a moving stage mounted at the biochip, wherein the moving stage is sized and configured to accurately position each of said reagent wells under said actuator, and position said first reaction well above or below said detector; and
(d) a processor configured to control said moving stage and process the optical properties.

2. The apparatus of claim 1, further comprising a vacuum suction connected to said first reaction well and remove the fluid away from said first reaction well.

3. The apparatus of claim 1, further comprising a magnetic field generator situated in proximity of said first reaction well, wherein the generator has on and off switching mechanisms.

4. The apparatus of claim 3, wherein said magnetic field generator is an external magnet or a built-in electromagnetic element.

5. The apparatus of claim 1, wherein at least one of self-contained reagents comprises magnetic particles.

6. The apparatus of claim 1, wherein said moving stage is an X-Y translation stage or a rotational stage.

7. The apparatus of claim 1, further comprising a heating and cooling element for controlling a temperature of the fluid.

8. The apparatus of claim 7, wherein the moving stage is further configured to accurately position the first reaction well above said heating and cooling element.

9. The apparatus of claim 7, wherein said heating and cooling element is a Peltier thermal heat pump.

10. The apparatus of claim 1, wherein said optical properties are chemiluminescence or bioluminescence signals.

11. The apparatus of claim 1, further comprising a light source located above or below the biochip, wherein the light source illuminates said first reaction well for light absorbance or fluorescence measurement.

12. A method for measuring optical properties of a fluid combining at least two reagents in a biochip system, the method comprising:
providing the biochip system having a biochip, a releasing actuator, a detector, a moving stage, and a processor, wherein said biochip comprises a plurality of patterned reagent wells with self-contained reagents that are connected to reaction wells via microchannels, the moving stage being mounted at the biochip, the moving stage being sized and configured to accurately position each of said reagent wells under said actuator;
releasing a first self-contained reagent from a first reagent well by positioning and activating the releasing actuator onto a first reagent well and transporting released first reagent to a first reaction well;
releasing a second self-contained reagent from a second reagent well by positioning and activating the releasing actuator onto a second reagent well and transporting released second reagent to the first reaction well;
moving the first reaction well to proximity of the detector using the moving stage;
measuring optical properties of fluid in said first reaction well using the detector; and
processing the optical properties of the fluid using the processor.

13. The method of claim 12, wherein the biochip system further comprises a vacuum suction connected to said first reaction well, said vacuum suction removing the fluid away from said first reaction well.

14. The method of claim 12, wherein the biochip system further comprises a magnetic field generator situated in proximity of said first reaction well, wherein said magnetic field generator has on and off switching mechanisms, and wherein said magnetic field generator is an external magnet or a built-in electromagnetic element.

15. The method of claim 12, wherein at least one of self-contained reagents comprises magnetic particles.

16. The method of claim 12, wherein said moving stage is an X-Y translation stage or a rotational stage.

17. The method of claim 12, wherein the biochip system further comprises a heating and cooling element for controlling a temperature of the fluid.

18. The method of claim 17, wherein the moving stage is further configured to accurately position the first reaction well above said heating and cooling element.

18. The method of claim 17, wherein said heating and cooling element is a Peltier thermal heat pump.

19. The method of claim 12, wherein said optical properties are chemiluminescence or bioluminescence signals.

20. The method of claim 12, wherein the biochip system further comprises a light source for illuminating said first reaction well for light absorbance or fluorescence measurement.
